(19) United States
(12) Patent Application Publication
(10) Pub. No.: US 2002/0127252 A1
(43) Pub. Date: Sep. 12, 2002

(54) Use of chroman derivatives in cosmetic or dermatological preparations

(76) Inventors: Klaus Kramer, Landau (DE); Hagen Jaedicke, Ludwigshafen (DE); Axel Jentzsch, Mannheim (DE)

Correspondence Address: KEIL & WEINKAUF, 1350 CONNECTICUT AVENUE, N.W., WASHINGTON, DC, 20036, US

(21) Appl. No.: 10/015,559

(22) Filed: Dec. 17, 2001

(30) Foreign Application Priority Data
Dec. 22, 2000 (DE) 10064818.5

(51) Int. Cl. A61K 31/353; A61K 7/00

(52) U.S. Cl. 424/401; 514/456

(57) ABSTRACT

The invention relates to the use of chroman derivatives of the formula I,

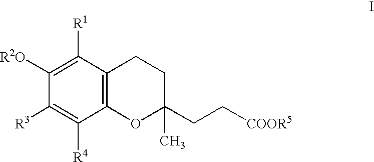

in which the substituents, independently of one another, have the following meanings:

R¹ is hydrogen, C₁-C₁₂-alkyl,

R² is hydrogen, C₁-C₁₂-acyl,

R³ and R⁴ are hydrogen, C₁-C₁₂-alkyl,

R⁵ is hydrogen, C₁-C₁₂-alkyl, aryl

in cosmetic or dermatological preparations.

The present invention relates to the use of chroman derivatives in cosmetic preparations. The invention also relates to cosmetic preparations having an effective content of chroman derivatives. In particular, the present invention relates to cosmetic preparations for prophylaxis against aging processes in the skin.

The human skin is subject to certain aging processes, some of which are to be attributed to intrinsic processes (chronoaging) and some of which are to be attributed to exogenous factors (environmental, e.g. photoaging). In addition, temporary and also permanent changes in the appearance of the skin can arise, such as acne, greasy or dry skin, keratoses, rosaceae, light-sensitive, inflammatory, erythematous, allergic or autoimmune reactions, such as dermatoses and photodermatoses.

Exogenous factors include, for example, sunlight or artificial radiation sources having a comparable spectrum, and compounds which can form as a result of the radiation, such as undefined reactive photoproducts, which may also be radical or ionic. These factors also include cigarette smoke and the reactive compounds present therein, such as ozone, free radicals, for example the hydroxyl radical, singlet oxygen and other reactive oxygen or nitrogen compounds which interfere with the natural physiology or morphology of the skin.

The effect of these factors may result inter alia in direct damage to the DNA of the skin cells, and to the collagen, elastin or glycosaminoglycan molecules of the extracellular matrix which are responsible for the strength of the skin. Moreover, signal transduction chains may be affected, resulting in the activation of matrix-degrading enzymes. Important representatives of these enzymes are the matrix metalloproteinases (MMPs, e.g. collagenases, gelatinases, stromelysines), the activity of which is additionally regulated by TIMPs (tissue inhibitor of matrix metalloproteinases).

The consequences of the abovementioned aging processes are thinning of the skin, weaker meshing of epidermis and dermis, reduction in cell number and in supplying blood vessels. This leads to the formation of fine lines and wrinkles, the skin becomes leathery, and pigmentation disorders may arise.

The same factors also act on hair, likewise leading to possible damage. The hair becomes brittle, less elastic and dull. The surface structure of the hair is damaged.

Cosmetic or dermatological care products having properties which are intended to counteract the processes described or comparable processes, or to alleviate or reverse the harmful consequences thereof are frequently notable for the following specific properties—they have a free-radical scavenging action, an antioxidative action, antiinflammatory action or moisturizing action. They prevent or reduce inter alia the activity of the matrix-degrading enzymes or regulate the further synthesis of collagen, elastin or proteoglycans.

The use of antioxidants or free-radical scavengers in cosmetic preparations is sufficiently known per se. For example, the use of the antioxidative vitamin E in sunscreen preparations is already prior art (e.g. De Polo, KF “A Short Textbook of Cosmetology” 1998). Nevertheless, the effect which is achieved even here falls a long way short of the desired effect.

Because of the ever growing need for cosmetic active ingredients for the preventive treatment of human skin and human hair against aging processes and harmful environmental effects, the object of the present invention was to provide novel active ingredients for cosmetic use which are to display the cosmetic effects already mentioned in the introduction, be sufficiently oxidation-stable and photostable and be able to be formulated easily. The resulting cosmetic preparations should also have as low an irritation potential for the skin as possible, they should positively influence water binding in the skin, increase the elasticity of the skin and therefore effect smoothing of the skin. Moreover, when applied to the skin, they should produce a pleasant feel on the skin.

We have found that this object is achieved by the use of chroman derivatives of the formula I,

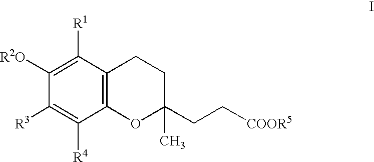

in which the substituents, independently of one another, have the following meanings:

R¹ is hydrogen, C₁-C₁₂-alkyl,

R² is hydrogen, C₁-C₁₂-acyl,

R³ and R⁴ are hydrogen, C₁-C₁₂-alkyl,

R⁵ is hydrogen, C₁-C₁₂-alkyl, aryl

in cosmetic or dermatological preparations.

The chroman derivatives of the formula I used according to the invention may either be enantiomerically pure compounds or racemic mixtures.

Alkyl radicals for R¹ and R³ to R⁵ which may be mentioned are branched or unbranched C₁-C₁₂-alkyl chains, such as methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl, n-decyl, n-undecyl and n-dodecyl.

Preferred alkyl radicals of C₁-C₃-alkyl chains, particularly preferably methyl, ethyl, n-propyl and 1-methylethyl.

Aryl for R⁵ is understood as meaning aromatic rings or ring systems having 6 to 18 carbon atoms in the ring system, for example phenyl or naphthyl, which may optionally be substituted by one or more radicals such as halogen, e.g. fluorine, chlorine or bromine, cyano, nitro, amino, C₁-C₄-alkylamino, C₁-C₄-dialkylamino, hydroxyl, C₁-C₄-alkyl, C₁-C₄-alkoxy or other radicals. Preference is given to phenyl, methoxyphenyl and naphthyl.

Acyl radicals for R² are to be understood as meaning branched or unbranched, saturated or unsaturated, optionally polyunsaturated, C₁-C₁₂-acyl chains.

Examples thereof are acyl radicals of formic acid, acetic acid, propionic acid, n-butyric acid, isobutyric acid, sorbic acid, n-valeric acid, isovaleric acid, caproic acid, caprylic acid, capric acid and undecanoic acid.

Preference is given to C₁-C₃-acyl radicals, and particular preference is given to radicals of acetic acid and propionic acid.

Very particular preference is given to 2,5,7,8-tetramethyl-2-(β-carboxyethyl)-6-hydroxychroman [α-CEHC] of the formula Ia

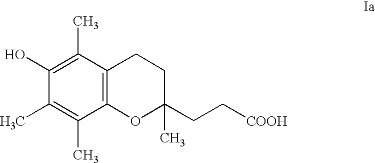

α-CEHC is known as a natural degradation product of α-tocopherol. This is found both in the urine and also in the blood serum of humans (Schultz et al., Am. J. Clin. Nutr., 62(suppl.), 1527S-1534S, 1995; W. Stahl et al., Analytical Biochemistry, 275, 254-259, 1999).

The use of α-CEHC in cosmetics was not hitherto known—either for preventing or for treating skin damage.

It was therefore surprising that using chroman derivatives of the formula I, in particular using α-CEHC, both a preventive effect against skin damage, in particular a preventive effect against aging processes of the human skin, and also a cosmetic effect against skin damage which has already occurred can be achieved.

The use according to the invention of chroman derivatives of the formula I in cosmetic preparations offers, inter alia, protection against damage caused directly or indirectly by UV irradiation or by processes triggered by reactive compounds, such as e.g.

skin aging,

loss of skin moisture,

loss of skin elasticity,

formation of lines or wrinkles or

pigment disorders or age spots.

The present invention further relates to the use of the abovementioned preparations for the cosmetic prevention of undesired changes in the appearance of the skin, such as e.g.

acne or greasy skin,

keratoses,

rosaceae,

light-sensitive, inflammatory, erythematous, allergic or autoimmune reactions.

In addition, the chroman derivatives of the formula I used according to the invention in hair cosmetic preparations offer protection against premature aging processes of human hair and can thus be used as active ingredients in the cosmetic treatment of brittle, dull and inelastic hair.

The invention further relates to the use of chroman derivatives of the formula I for stabilizing cosmetic and dermatological preparations or for stabilizing other active ingredients which are present in such preparations, e.g. against harmful oxidation processes or microbial decay.

The invention further relates to cosmetic preparations for protecting the human epidermis or human hair, which comprise, in a cosmetically suitable carrier, a cosmetically effective amount of at least one of the compounds of the formula I

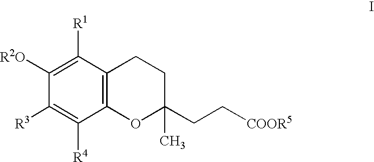

in which the radicals R¹ to R⁵ have the meanings given at the outset.

The cosmetic preparations according to the invention also serve, however, for the calming of sensitive and irritated skin, for the preventive regulation of the synthesis of collagen, hyaluronic acid and elastin, stimulation of DNA synthesis, in particular in the case of deficient or hypoactive skin states, regulation of the transcription and translation of matrix-degrading enzymes, in particular MMPs, increase in cell renewal and regeneration of the skin, increase in endogenous skin protection and repair mechanisms for DNA, lipids and/or proteins.

The cosmetic formulations according to the invention can have the customary composition and be used for the preventive treatment, care and cleansing of the skin and of the hair and as make-up product in cosmetics. They generally comprise 0.01 to 30% by weight, preferably 0.01% by weight to 15% by weight, particularly preferably 0.05 to 5% by weight, very particularly preferably 0.1 to 2% by weight, of at least one of the compounds of the formula I.

For use, the cosmetic preparations according to the invention are applied in the manner customary for cosmetics to the skin and/or the hair in a sufficiently effective amount.

A preparation according to the invention can, for example, be a solution, an anhydrous preparation, an emulsion or microemulsion of the water-in-oil type or of the oil-in-water type, a multiple emulsion, for example of the water-in-oil-in-water type, a gel, a solid stick, an ointment, an aerosol or else an aqueous system or a surfactant preparation for the cleansing of skin and/or hair.

It is also advantageous to administer the compounds of the formula I in encapsulated form, e.g. as cellulose capsules, in gelatin, wax matrices, with cyclodextrins or liposomally encapsulated.

The cosmetic and dermatological preparations according to the invention can comprise cosmetic auxiliaries as are customarily used in such preparations, e.g. preservatives, bactericides, perfumes, antifoams, dyes, pigments which have a coloring action, thickeners, moisturizing and/or humectant substances, fats, oils, waxes or other customary constituents of a cosmetic or dermatological formulation, such as alcohols, polymers, foam stabilizers, electrolytes, organic solvents or silicone derivatives.

An additional content of antioxidants is generally preferred. According to the invention, favorable antioxidants are all antioxidants which are customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very small tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, transstilbene oxide) and the derivatives (salts, esters, ethers, sugars, nucleotides, nucleosides, peptides and lipids) of said active ingredients which are suitable according to the invention.

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are the antioxidant(s), it is advantageous to choose their respective concentrations from the range 0.001-10% by weight, based on the total weight of the formulation. If vitamin A or vitamin A derivatives or carotenes or derivatives thereof are the antioxidant(s), it is advantageous to choose their respective concentrations from the range 0.001-10% by weight, based on the total weight of the formulation.

The lipid phase can advantageously be chosen from the following groups of substances:

mineral oils, mineral waxes

oils, such as triglycerides of capric or of caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids; alkyl benzoates;

silicone oils, such as dimethylpolysiloxanes, diethylpolysiloxanes, diphenylpolysiloxanes and mixed forms thereof.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids of chain length from 3 to 30 carbon atoms, from the group of esters of aromatic carboxylic acids and saturated and/or unsaturated, branched and/or unbranched alcohols of chain length from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can furthermore advantageously be chosen from the group of branched and unbranched hydrocarbons and hydrocarbon waxes, silicone oils, dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, be advantageously chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Also, any mixtures of such oil and wax components can be advantageously used for the purposes of the present invention. It may also be advantageous in some instances to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicapryl ether.

Particularly advantageous mixtures comprise C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can further advantageously have a content of cyclic or linear silicone oils or consist entirely of such oils, although it is preferred, apart from the silicone oil or silicone oils, to use an additional content of other oil phase components.

Cyclomethicone (octamethylcyclotetrasiloxane) is advantageously used as the silicone oil to be used according to the invention. However, other silicone oils are also to be used advantageously for the purposes of the present invention, for example, hexamethylcyclotrisiloxane, polydimethylsiloxane, poly(methylphenylsiloxane).

Other particularly advantageous mixtures comprise cyclomethicone and isotridecyl isononanoate, or cyclomethicone and 2-ethylhexyl isostearate.

The aqueous phase of the preparations according to the invention optionally advantageously comprises

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and in particular one or more thickeners, which can advantageously be chosen from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of Carbopols, for example Carbopols grades 980, 981, 1382, 2984, 5984, each individually or in combination.

It is further advantageous to use additional oil-soluble organic UVA filters and/or UVB filters in the lipid phase and/or water-soluble organic UVA filters and/or UVB filters in the aqueous phase, the total amount of filter substances being, for example, 0.1% by weight to 30% by weight, preferably 0.5 to 15% by weight, in particular 1 to 10% by weight, based on the total weight of the preparations, in order to make available cosmetic preparations which protect the skin from the entire region of ultraviolet radiation.

Examples of light protection agents which can be used alone or in mixtures together with the compounds of the formula I are:

TABLE 1

| | | CAS No. |
|---|---|---|
| No. | Substance | (= acid) |
| 1 | 4-aminobenzoic acid | 150-13-0 |
| 2 | 3-(4′-trimethylammonium)-benzylidenebor- | 52793-97-2 |
| | nan-2-one methylsulfate | |
| 3 | 3,3,5-trimethyl-cyclohexyl salicylate | 118-56-9 |
| | (homosalate) | |
| 4 | 2-hydroxy-4-methoxy-benzophenone (oxybenzone) | 131-57-7 |
| 5 | 2-phenylbenzimidazole-5-sulfonic acid and its | 27503-81-7 |
| | potassium, sodium and triethanolamine salts | |
| 6 | 3,3′-(1,4-phenylenedimethine)-bis(7,7-dimethyl- | 90457-82-2 |
| | 2-oxobicyclo[2.2.1]heptane-1-methanesulfonic | |
| | acid) and its salts | |
| 7 | polyethoxyethyl 4-bis(polyethoxy)aminobenzoate | 113010-52-9 |
| 8 | 2-ethylhexyl 4-dimethylaminobenzoate | 21245-02-3 |
| 9 | 2-ethylhexyl salicylate | 118-60-5 |
| 10 | isoamyl 4-methoxycinnamate | 71617-10-2 |
| 11 | 2-ethylhexyl 4-methoxycinnamate | 5466-77-3 |
| 12 | 2-hydroxy-4-methoxybenzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-benzylidenebornan-2-one | 16087-24-8 |
| 15 | 1-(4′-isopropylphenyl)-3-phenylpro- | 63260-25-9 |
| | pane-1,3-dione | |
| 16 | 4-isopropylbenzyl salicylate | 94134-93-7 |
| 17 | 2,4,6-trianilino(o-carbo-2′-ethylhexyl-1′-oxy) | 88122-99-0 |
| | 1,3,5-triazine | |
| 18 | 3-imidazol-4-ylacrylic acid and its ethyl ester | 104-98-3 |
| 19 | menthyl o-aminobenzoate or: | 134-09-8 |
| | 5-methyl-2-(1-methylethyl)-2-aminobenzoate | |
| 20 | glyceryl p-aminobenzoate or: | 136-44-7 |
| | 1-glyceryl 4-aminobenzoate | |
| 21 | 2,2′-dihydroxy-4-methoxybenzophenone (dioxyben- | 131-53-3 |
| | zone) | |
| 22 | 2-hydroxy-4-methoxy-4-methylbenzophenone - | 1641-17-4 |
| | (mexenone) | |
| 23 | triethanolamine salicylate | 2174-16-5 |
| 24 | dimethoxyphenylglyoxalic acid or: sodium | 4732-70-1 |
| | 3,4-dimethoxyphenylglyoxalate | |
| 25 | 3-(4′-sulfobenzylidene)bornan-2-one and its | 56039-58-8 |
| | salts | |
| 26 | 2,2′,4,4′-tetrahydroxybenzophenone | 131-55-5 |
| 27 | 2,2′-methylenebis[6-(2H-benzotria- | 103597-45-1 |
| | zol-2-yl)-4-(1,1,3,3-tetramethylbutyl)phenol] | |
| 28 | 2,2′-(1,4-phenylene)bis-1H-benzimida- | 180898-37-7 |
| | zole-4,6-disulfonic acid, Na salt | |
| 29 | 2,4-bis[4-(2-ethylhexyloxy)-2-hydroxy]phe- | 187393-00-6 |
| | nyl-6-(4-methoxyphenyl)-(1,3,5)-triazine | |
| 30 | 3-(4-methylbenzylidene)camphor | 36861-47-9 |
| 31 | polyethoxyethyl 4-bis(polyethoxy)paraamino- | 113010-52-9 |
| | benzoate | |
| 32 | 2,4-dihydroxybenzophenone | 131-56-6 |
| 33 | 2,2′-dihydroxy-4,4′-dimethoxybenzophe- | 3121-60-6 |
| | none-5,5′-disodium sulfonate | |

Other light protection agents which can be combined are, inter alia, the following compounds:

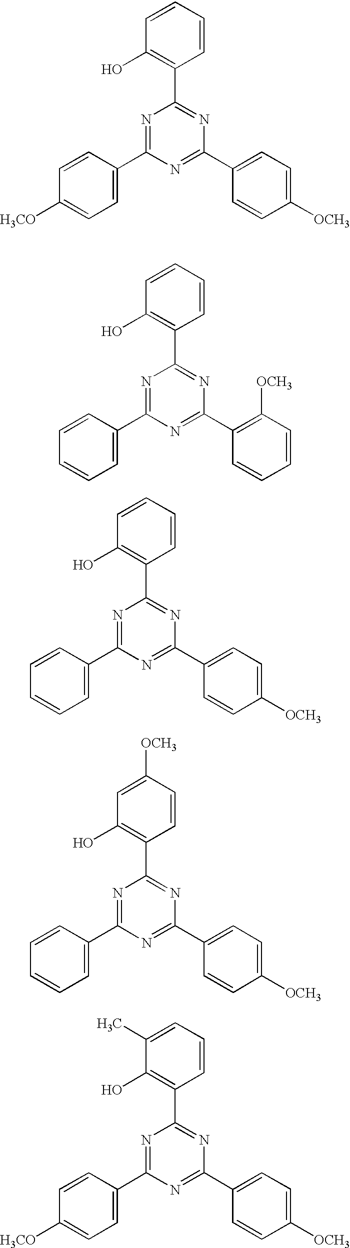

The list of said UV filters which can be used in combination with the chroman derivatives used according to the invention is not of course intended to be limiting.

Cosmetic and dermatological preparations having an effective content of at least one chroman derivative of the formula I can also contain inorganic pigments which are normally used in cosmetics for protecting the skin against UV rays. These are oxides of titanium, zinc, zirconium, silicon, manganese, cerium and mixtures thereof, and modifications in which the oxides are the active agents. Particular preference is given to pigments based on titanium dioxide.

Cosmetic and dermatological preparations for protecting the hair according to the invention are, for example, shampoos, preparations which are applied to the hair when rinsing the hair before or after shampooing, before or after permanent waving, before or after coloring or bleaching, preparations for blow-drying or setting the hair, preparations for coloring or bleaching, a styling and treatment lotion, a hairspray or a permanent wave composition.

The cosmetic and dermatological preparations comprise active ingredients and auxiliaries which are usually used for this type of preparation for hair care and hair treatment. Auxiliaries include preservatives, surfactants, antifoams, thickeners, emulsifiers, fats, oils, waxes, organic solvents, bactericides, perfumes, dyes or pigments whose task is to color the hair or the cosmetic or dermatological preparation itself, electrolytes and anti-grease substances.

For the purposes of the present invention, the term “electrolytes” means water-soluble alkali metal, ammonium, alkaline earth metal (including magnesium) and zinc salts of inorganic anions and any mixtures of such salts, it being necessary to ensure that these salts are pharmaceutically or cosmetically safe.

The anions according to the invention are preferably chosen from the group consisting of chlorides, sulfates and hydrogensulfates, phosphates, hydrogenphosphates and linear and cyclic oligophosphates and carbonates and hydrogencarbonates.

If the cosmetic or dermatological preparations are in the form of a lotion which is rinsed out and applied, for example, before or after bleaching, before or after shampooing, between two shampooing steps, before or after permanent waving, they are, for example, aqueous or aqueous-alcoholic solutions optionally comprising surface-active substances in a concentration of between 0.1 and 10% by weight, preferably between 0.2 and 5% by weight.

These cosmetic or dermatological preparations can also be in the form of aerosols with the auxiliaries customarily used for this purpose.

A cosmetic preparation in the form of a lotion which is not rinsed out, in particular a lotion for setting the hair, a lotion which is used for blow drying the hair, a styling and treatment lotion, is generally in the form of an aqueous, alcoholic or aqueous-alcoholic solution, and contains at least one cationic, anionic, nonionic or amphoteric polymer or also mixtures thereof, and also at least one chroman derivative of the formula I in an effective concentration. The amount of polymers used is, for example, between 0.1 and 10% by weight, preferably between 0.1 and 3% by weight.

Cosmetic preparations for treating and caring for the hair which comprise at least one chroman derivative of the formula I can be in the form of emulsions which are of the nonionic or anionic type. Nonionic emulsions comprise, in addition to water, oils or fatty alcohols which may, for example, also be polyethoxylated or polypropoxylated, or also mixtures of the two organic components. These emulsions optionally comprise cationic surface-active substances.

According to the invention, cosmetic preparations for treating and caring for the hair can be in the form of gels which, in addition to an effective content of at least one chroman derivative of the formula I and solvents usually used for this purpose, preferably water, also contain organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, for example aluminum silicates such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The amount of chroman derivative of the formula I in a composition intended for hair is preferably from 0.01% by weight to 30% by weight, preferably from 0.05% by weight to 5% by weight, in particular from 0.1% by weight to 2% by weight, based on the total weight of the composition.

Aqueous cosmetic cleansers according to the invention or low-water or water-free cleanser concentrates intended for aqueous cleansing may contain anionic, nonionic and/or amphoteric surfactants, for example

conventional soaps, e.g. fatty acid salts of sodium

alkyl sulfates, alkyl ether sulfates, alkanesulfonates and alkylbenzenesulfonates

sulfoacetates

sulfobetaines

sarcosinates

amidosulfobetaines

sulfosuccinates

sulfosuccinic acid monoesters

alkyl ether carboxylates

protein-fatty acid condensates

alkylbetaines and amidobetaines

fatty acid alkanolamides

polyglycol ether derivatives.

Cosmetic preparations which are cosmetic cleansing preparations for the skin can be in liquid or solid form. In addition to at least one chroman derivative of the formula I, they preferably comprise at least one anionic, nonionic or amphoteric surface-active substance or mixtures thereof, if desired one or more electrolytes and auxiliaries customarily used for this purpose. The surface-active substance can be present in the cleansing preparations in a concentration of between 1 and 94% by weight, based on the total weight of the preparations.

Cosmetic preparations in the form of a shampoo preferably comprise, in addition to an effective content of at least one chroman derivative of the formula I, at least one anionic, nonionic or amphoteric surface-active substance or mixtures thereof, if desired an electrolyte according to the invention and auxiliaries which are customarily used for this purpose. The surface-active substance can be present in the shampoo in a concentration of between 1% by weight and 94% by weight.

The compositions according to the invention comprise, in addition to the aforementioned surfactants, water and, when required, the additives customary in cosmetics, for example perfume, thickeners, dyes, deodorants, antimicrobial substances, refatting agents, complexing agents and sequestering agents, pearlizing agents, plant extracts, vitamins, active ingredients and the like.

The present invention also covers a cosmetic method of protecting the skin and hair against oxidative or photooxidative processes which comprises applying a cosmetic composition which comprises an effective concentration of at least one chroman derivative of the formula I in a sufficient quantity to the skin or hair.

The present invention likewise also covers a method of protecting cosmetic or dermatological preparations against oxidation or photooxidation, these preparations being, for example, preparations for the treatment and care of hair, in particular hair colorants, hairsprays, shampoos, color shampoos, and also make-up products such as, for example, nail varnishes, lipsticks, foundations, washing and shower preparations, creams for the treatment or care of skin or all other cosmetic preparations whose constituents may bring with them stability problems because of oxidation or photooxidation during storage, wherein the cosmetic preparations have an effective content of at least one chroman derivative of the formula I.

The examples below illustrate in more detail compositions of cosmetic formulations which comprise chroman derivatives of the formula I.

### EXAMPLE 1

Soft Skin Fluid

| | Mass content |
|---|---|
| | (% by wt.) |
| Ceteareth-6 and Stearyl Alcohol | 2.50 |
| Ceteareth-25 | 2.50 |
| Hydrogenated Cocoglycerides | 1.50 |
| PEG-40 Dodecyl Glycol Copolymer | 3.00 |
| Dimethicone | 3.00 |
| Phenethyl Dimethicone | 2.00 |
| Cyclomethicone | 1.00 |
| Cetearyl Octanoate | 5.00 |
| Avocado Oil | 1.00 |
| Sweet Almond Oil | 2.00 |
| Wheat Germ Oil | 0.80 |
| α-CEHC | 0.20 |
| Panthenol USP | 1.00 |
| Phytantriol | 0.20 |
| Tocopheryl Acetate | 0.30 |
| Propylene Glycol | 5.00 |
| Fragrance | q.s. |
| Preservative | q.s. |
| Aqua | ad 100 |

### EXAMPLE 2

Hand Protection Cream

| | Mass content |
|---|---|
| | (% by wt.) |
| Cetearyl Alcohol | 1.00 |
| Glyceryl Stearate | 1.50 |
| Stearyl Alcohol | 1.50 |
| Cetyl Palmitate | 2.00 |
| Tocopheryl Acetate | 0.50 |
| Dimethicone | 8.00 |
| Ceteareth-6 and Stearyl Alcohol | 3.00 |
| Octyl Methoxycinnamate | 5.00 |
| Propylene Glycol | 8.00 |
| Panthenol | 1.00 |
| Evening Primrose Oil | 3.00 |
| PEG-7 Hydrogenated Castor Oil | 6.00 |
| Glyceryl Oleate | 1.00 |
| Phenethyl Dimethicone | 3.00 |
| Beeswax | 1.50 |
| Locust Bean Gum | 0.80 |
| Silk powder | 0.80 |
| Preservative | q.s. |
| Fragrance | q.s. |
| Borax | 0.10 |
| α-CEHC | 0.10 |
| Aqua | ad 100 |

### EXAMPLE 3

Sun Care Lotion

| | Mass content |
|---|---|
| | (% by wt.) |
| PEG-7 Hydrogenated Castor Oil | 6.00 |
| PEG-40 Hydrogenated Castor Oil | 0.50 |
| Isopropyl Palmitate | 7.00 |
| PEG-45/Dodecyl Glycol Copolymer | 2.00 |
| Jojoba Oil | 3.00 |
| Magnesium Stearate | 0.60 |
| Octyl Methoxycinnamate | 8.00 |
| C₁₂₋₁₅ Alkyl Benzoate | 5.00 |
| Titanium Dioxide | 4.00 |
| Propylene Glycol | 5.00 |
| EDTA | 0.20 |
| Preservative | q.s. |
| Sodium Ascorbyl Phosphate | 1.00 |
| Tocopheryl Acetate | 0.50 |
| α-CEHC | 0.20 |
| Fragrance | q.s. |
| Aqua | ad 100 |

### EXAMPLE 4

Multiple Emulsion

| | Mass content |
|---|---|
| | (% by wt.) |
| Mineral Oil | 7.50 |
| Cetearyl Octanoate | 2.50 |
| Aluminum Stearate | 0.25 |
| Magnesium Stearate | 0.25 |
| Microcrystalline Wax H | 0.50 |
| Cetearyl Alcohol | 1.00 |
| Lanolin Alcohol | 1.50 |
| Mineral Alcohol and Lanolin Alcohol | 1.50 |
| PEG-7 Hydrogenated Castor Oil | 0.75 |
| PEG-45/Dodecyl Glycol Copolymer | 2.00 |
| Ceteareth-6 and Stearyl Alcohol | 2.00 |
| Ceteareth-25 | 2.00 |
| Trilaureth-4 Phosphate | 1.00 |
| Hydroxyethylcellulose | 0.20 |
| Propylene Glycol | 7.50 |
| Magnesium Sulfate | 0.25 |
| α-CEHC | 0.10 |
| Water | ad 100 |

### EXAMPLE 5

Microemulsion

| | Mass content |
|---|---|
| | (% by wt.) |
| Ceteareth-25 | 13.00 |
| PEG-7 Glyceryl Cocoate | 20.00 |
| Octyl Dodecanol | 5.00 |
| Preservative | q.s. |
| α-CEHC | 0.20 |
| Aqua | ad 100 |

### EXAMPLE 6

Liposome gel

| | Mass content |
|---|---|
| | (% by wt.) |
| PEG-40 Hydrogenated Castor Oil | 1.00 |
| Bisabolol rac. | 0.10 |
| Propylene Glycol | 8.00 |
| Panthenol | 0.50 |
| Tocopheryl Acetate and Polysorbate 80 | 3.00 |
| and Caprylic/Capric Triglyceride | |
| and Lecithin | |
| Preservative | q.s. |
| Perfume | q.s. |
| Carbomer | 0.50 |
| α-CEHC | 0.20 |
| Triethanolamine | 0.70 |
| Aqua | ad 100 |

### EXAMPLE 7

Blunted Oil Gel

| | Mass content |
|---|---|
| | (% by wt.) |
| Silica | 5.00 |
| Dimethicone | 10.00 |
| Cetearyl Octanoate | 40.00 |
| Caprylic/Capric Triglyceride | 8.00 |
| Phenethyl Dimethicone | 2.00 |
| Mineral Oil | 28.50 |
| Sweet Almond Oil | 5.00 |
| Phytantriol | 0.30 |
| α-CEHC | 0.10 |
| Tocopherol | 0.50 |
| Fragrance | 1.00 |

### EXAMPLE 8

Oil Gel

| | Mass content |
|---|---|
| | (% by wt.) |
| Silica | 5.00 |
| Dimethicone | 10.00 |
| Cetearyl Octanoate | 30.00 |
| Caprylic/Capric Triglyceride | 10.00 |
| Isopropyl Myristate | 5.00 |
| Phenethyl Dimethicone | 5.00 |
| Mineral Oil | 28.20 |
| Jojoba Oil | 5.00 |
| Phytantriol | 0.30 |
| α-CEHC | 0.30 |
| Tocopherol | 0.50 |
| Fragrance | 1.00 |

### EXAMPLE 9

Sun Care Lip Protection Stick

| | Mass content |
|---|---|
| | (% by wt.) |
| Beeswax | 12.00 |
| Hydrogenated Cocoglycerides | 5.00 |
| Ricinus Oil | 40.00 |
| Isopropyl Palmitate | 10.00 |
| Mineral Oil | 10.00 |
| Candelilla Wax | 8.00 |
| Phenethyl Dimethicone | 5.00 |
| α-CEHC | 0.20 |
| Petrolatum | 5.00 |
| Benzophenone-3 | 5.00 |

### EXAMPLE 10

| | Mass content |
|---|---|
| Cooling Body Splash | (% by wt.) |
| PEG-40 Hydrogenated Castor Oil | 2.00 |
| Menthyl Lactate | 0.20 |
| Alcohol | 5.00 |
| PEG-7 Glyceryl Cocoate | 2.00 |
| Witch Hazel | 5.00 |
| Allantoin | 0.10 |
| Bisabolol rac. | 0.20 |
| Propylene Glycol | 5.00 |
| Panthenol USP | 0.50 |
| Lactic Acid (80% strength) | 0.20 |
| α-CEHC | 0.20 |
| Fragrance | q.s. |
| Aqua | ad 100 |

### EXAMPLE 11

| | Mass content |
|---|---|
| Make-up | (% by wt.) |
| Ceteareth-6 and Stearyl Alcohol | 9.00 |
| Dimethicone | 5.00 |
| Cetearyl Octanoate | 8.00 |
| Macadamia Nut Oil | 5.00 |
| Propylene Glycol | 5.00 |
| Sicovit White E 171 | 8.00 |
| Sicomet Brown 70 13E 3717 | 2.00 |
| α-CEHC | 0.20 |
| Fragrance | q.s. |
| Benzophenone-3 | 5.00 |
| Aqua | ad 100 |

### EXAMPLE 12

| | Mass content |
|---|---|
| Fluid Make-up | (% by wt.) |
| Ceteareth-6 and Stearyl Alcohol | 7.00 |
| Ceteareth-25 | 5.00 |
| Dimethicone | 5.00 |
| Cetearyl Octanoate | 8.00 |
| Macadamia Nut Oil | 5.00 |
| Propylene Glycol | 5.00 |
| Sicovit White E 171 | 8.00 |
| Sicomet Brown 70 13E 3717 | 1.00 |
| α-CEHC | 0.10 |
| Fragrance | q.s. |
| Benzophenone-3 | 5.00 |
| Aqua | ad 100 |

### EXAMPLE 13

| | Mass content |
|---|---|
| Sun Care Oil | (% by wt.) |
| Cetearyl Octanoate | 40.00 |
| Caprylic/Capric Triglyceride | 28.70 |
| Evening Primrose Oil | 3.00 |
| Macadamia Nut Oil | 5.00 |
| Isopropyl Palmitate | 5.00 |
| Dimethicone | 3.00 |
| Octyl Methoxycinnamate | 8.00 |
| Octocrylene | 5.00 |
| Benzophenone-3 | 2.00 |
| Phytantriol | 0.10 |
| α-CEHC | 0.10 |
| Tocopheryl Acetate | 0.20 |
| Fragrance | q.s. |

### EXAMPLE 14

| | Mass content |
|---|---|
| Facial Scrub Cleanser | (% by wt.) |
| Cocoamidopropyl Betaine | 5.00 |
| Potassium Coco-Hydrolyzed | 8.00 |
| Animal Protein | |
| PEG-40 Hydrogenated Castor Oil | 2.00 |
| Polyquaternium-44 | 7.70 |
| Bisabolol rac. | 0.20 |
| Panthenol | 1.00 |
| Fragrance | 0.50 |
| Hydroxyethyl Cellulose | 2.00 |
| α-CEHC | 0.50 |
| Propylene Glycol | 5.00 |
| Jojoba Wax | 3.00 |
| Water | ad 100 |

### EXAMPLE 15

| | Mass content |
|---|---|
| Conditioner | (% by wt.) |
| Ceteareth-6 and Stearyl Alcohol | 2.00 |
| Ceteareth-25 | 1.00 |
| Cetearyl Octanoate | 6.00 |
| Ceteareth-3 | 2.00 |
| Cetearyl Alcohol | 6.00 |
| Phytantriol | 1.00 |
| Propylene Glycol | 5.00 |
| Polyquaternium-11 | 5.00 |
| Panthenol | 1.00 |
| Retinyl Acetate | 0.50 |
| Fragrance | q.s. |
| α-CEHC | 0.50 |
| Preservative | q.s. |
| Aqua | ad 100 |

### EXAMPLE 16

| | Mass content |
|---|---|
| Hair Wax | (% by wt.) |
| Polyethylene Glycol-6 | 30.00 |
| Polyethylene Glycol-75 | 45.00 |
| Paraffinum Liquidum | 0.50 |
| PEG-40 Hydrogenated Castor Oil | 1.00 |
| Glycerin | 15.00 |
| Benzophenone-3 | 2.00 |
| Phytantriol | 0.10 |
| α-CEHC | 0.30 |
| Fragrance | q.s. |
| Aqua | ad 100 |

### EXAMPLE 17

| | Mass content |
|---|---|
| Anti-Dandruff Hair Tonic | (% by wt.) |
| Alcohol | 45.00 |
| Aloe Vera (10-fold conc.) | 1.00 |
| Panthenol | 1.00 |
| Tocopheryl Acetate | 0.50 |
| PEG-40 Hydrogenated Castor Oil | 0.50 |
| Allantoin | 0.10 |
| Hydrolyzed Animal Protein | 1.50 |
| 1-(4-Chlorophenoxy)-1-(1H-imi- | 0.30 |
| dazolyl)-3,3-dimethyl-2-butanone | |
| Fragrance | 0.10 |
| α-CEHC | 0.40 |
| Aqua | ad 100 |

### EXAMPLE 18

| | Mass content |
|---|---|
| Foot Deo Spray | (% by wt.) |
| PEG-40 Hydrogenated Castor Oil | 0.80 |
| Alcohol | 20.00 |
| Farnesol | 0.12 |
| Menthyl Lactate | 0.08 |
| 1,2-Propylene Glycol | 3.20 |
| Benzophenone-4 | 1.20 |
| PEG-7 Glyceryl Cocoate | 0.80 |
| Fragrance | q.s. |
| α-CEHC | 0.40 |
| Butane | 60.00 |
| Aqua | ad 100 |

### EXAMPLE 19

| | Mass content |
|---|---|
| Hair Spray | (% by wt.) |
| Aminomethyl Propanol | 0.40 |
| Dimethicone Copolyol | 0.03 |
| Alcohol | 43.67 |
| Pentane | 14.20 |
| Acrylates/Acrylamide Copolymer | 3.40 |
| Fragrance | q.s. |
| α-CEHC | 0.30 |
| Butane | 2.40 |
| Isobutane | ad 100 |

We claim:

1. The use of chroman derivatives of the formula I, 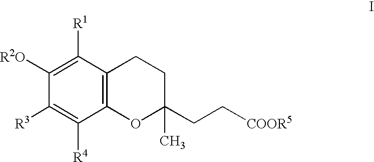
in which the substituents, independently of one another, have the following meanings:
R¹ is hydrogen, C₁-C₁₂-alkyl,
R² is hydrogen, C₁-C₁₂-acyl,
R³ and R⁴ are hydrogen, C₁-C₁₂-alkyl,
R⁵ is hydrogen, C₁-C₁₂-alkyl, aryl
in cosmetic or dermatological preparations.

2. The use of chroman derivatives as claimed in claim 1, in which
R¹ is hydrogen, C₁-C₃-alkyl,
R² is hydrogen, C₁-C₃-acyl,
R³ and R⁴ are C₁-C₃-alkyl and
R⁵ is hydrogen, C₁-C₃-alkyl.

3. The use of a chroman derivative as claimed in claim 1 having the formula Ia

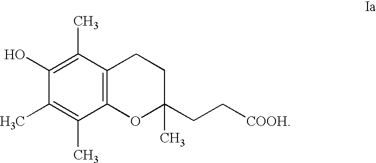

4. The use as claimed in any of claims 1 to 3 for prophylaxis against aging processes of the human skin.

5. The use as claimed in claim 4 for prophylaxis against dry skin, wrinkle formation and/or pigment disorders.

6. The use as claimed in any of claims 1 to 3 for prophylaxis against aging processes of human hair.

7. The use as claimed in any of claims 1 to 6, wherein at least one of the compounds of the formula I is present in an effective content in cosmetic preparations.

8. The use as claimed in claim 7, wherein at least one of the compounds of the formula I is present in concentrations of from 0.01 to 30% by weight, based on the total amount of the cosmetic preparation.

9. A cosmetic preparation for protecting the human epidermis or human hair, which comprises, in a cosmetically suitable carrier, a cosmetically effective amount of at least one of the compounds of the formula I 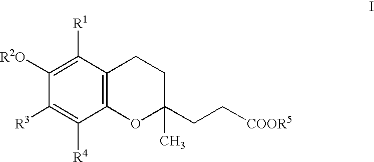
in which the substituents R¹ to R⁵ have the meanings defined according to either claim 1 or 2.

10. A cosmetic preparation as claimed in claim 9, comprising a cosmetically effective amount of the compound of the formula Ia [chemical structure]
